(19) United States
(12) Patent Application Publication
(10) Pub. No.: US 2013/0344178 A1
(43) Pub. Date: Dec. 26, 2013

(54) NATURAL SHAMPOO USING HERBAL MEDICINE MATURE DECOCTION HAVING A FUNCTION OF PREVENTING HAIR LOSS AND STIMULATING HAIR GROWTH, AND METHOD OF PREPARING THE SAME

(71) Applicant: Eun Joo KO, Seoul (KR)

(72) Inventor: Eun Joo KO, Seoul (KR)

(21) Appl. No.: 13/924,844

(22) Filed: Jun. 24, 2013

(30) Foreign Application Priority Data
Jun. 22, 2012 (KR) 10-2012-0067129

(51) Int. Cl. A61K 8/97; A61Q 7/00

(52) U.S. Cl. CPC A61K 8/97; A61Q 7/00
USPC 424/732

(57) ABSTRACT

A natural shampoo is provided. The natural shampoo is made using a herbal medicine mature decoction which is obtained by putting herbs including lotus roots, lotus leaves, red ginseng, Hippophae rhamnoides leaves and trunk, persimmon leaves, thuja orientalis, ginger, rosemary, black beans, black sesame, aloe, mulberry leaves, Orostachys japonicus, Angelica gigas, green tea, pine needles, mint, pleuropterus multflorus, blueberry, Acorus gramineus, and garlic in a maturing device, maturing the herbs in temperatures of 65 to 95° C. depending on the herb for 3 to 21 days, then pouring distilled water or soft water, and decocting the matured herbs mixed with the water.

### CROSS-REFERENCE TO RELATED PATENT APPLICATION

This application claims the benefit of Korean Patent Application No. 10-2012-0067129, filed on Jun. 22, 2012, in the Korean Intellectual Property Office, the disclosure of which is incorporated herein in its entirety by reference.

### BACKGROUND OF THE INVENTION

1. Field of the Invention

The present invention relates to a natural shampoo prepared by using Korean Patent No. 1128299, and more particularly, to a natural shampoo prepared by using a herbal medicine mature decoction which may be easy to be used by significantly reducing unpleasant smell of natural herbs, may be mixed with various natural herbs by reducing the attribute of the natural herbs of being separated from an carrier oil, may be stored for a long time by doubling elements having medical effects of natural herbs, may prevent loss of hair and stimulate hair growth by using a herbal medicine mature decoction of Korean Patent No. 1128299 owned by the applicant of the present application, and a method of preparing the same.

2. Description of the Related Art

Generally, a natural shampoo is a shampoo prepared without a synthetic surfactant and by adding a functional element such as a natural element and aroma oil in a home or in a small scale, which is contrasted with shampoos manufactured in a large scale in a factory in a current situation in which environmental friendliness and well-being are emphasized and harmfulness of the synthetic surfactant is being highlighted.

The natural shampoo may be broadly divided into a hot process (HP) shampoo and a cold process (CP) shampoo depending on the manufacturing method.

The HP shampoo is made by mixing caustic potash with oil in a high temperature. The shampoo has a good detergency, and the fragrance lasts for a long time. In order to make the HP shampoo, the elements should be heated in a water bath for 2 to 3 hours, or directly heated for 40 to 50 minutes, and thereafter, the heated elements should be matured in a normal temperature for 2 weeks. The CP shampoo is made by reacting caustic potash with oil in a low temperature and adding several additives. The CP shampoo is opaque, and a 6 week maturing period in a normal temperature is needed.

Such a natural shampoo does not contain synthetic perfume and artificial colorants, and moisturizing effects are good and harmfulness is less.

Further, the expiration date of the natural shampoo is only 6 months from the manufacturing date because preservatives are not added.

Further, recently, in order to add functionality to natural shampoos, some herbal medicine powders have been added to the shampoos.

The present applicant has also performed a lot of experiments for several years with an intention to add boiled or decocted herbal medicine to a natural shampoo because boiled or decocted herbal medicine is significantly more effective than powder, but many herbs except only a few herbs have not been mixed with carrier oil, the essential element for the natural shampoo, and thus the shampoo has not been made.

Therefore, it has been substantially impossible to make a natural shampoo which contains boiled or decocted herb medicines.

### SUMMARY OF THE INVENTION

The present invention provides a natural shampoo prepared by using a herbal medicine mature decoction which may be easy to be used by significantly reducing unpleasant smell of natural herbs, may be mixed with various natural herbs by reducing the attribute of the natural herbs of being separated from an carrier oil, may be stored for a long time by doubling elements having medical effects of natural herbs, may prevent loss of hair and stimulate hair growth by using a herbal medicine mature decoction of Korean Patent No. 1128299 owned by the applicant of the present application, and a method of preparing the same.

According to an aspect of the present invention, there is provided a natural shampoo made using a herbal medicine mature decoction which is obtained by putting herbs including lotus roots, lotus leaves, red ginseng, Hippophae rhamnoides leaves and trunk, persimmon leaves, thuja orientalis, ginger, rosemary, black beans, black sesame, aloe, mulberry leaves, Orostachys japonicus, Angelica gigas, green tea, pine needles, mint, pleuropterus multflorus, blueberry, Acorus gramineus, and garlic in a maturing device, maturing the herbs in temperatures of 65 to 95° C. depending on the herb for 3 to 21 days, then pouring distilled water or soft water, and decocting the matured herbs mixed with the water.

The herbal medicine mature decoction may be obtained by putting the herbs matured in the temperatures along with the distilled water or soft water in a decoction maker, then boiling the herbs mixed with the water by strong heat, then decocting the herbs mixed with the water by weak heat so as to make a first decoction, then pouring the first decoction in another vessel, then pouring distilled water or soft water in the decoction maker, then boiling the herbs mixed with the water by strong heat, then decoct the herbs mixed with the water by weak heat so as to make a second decoction, then removing the herbs from the second decoction, then mixing the first decoction with the second decoction, and then decocting the mixed decoction by weak heat.

According to another aspect of the present invention, there is provided a method of preparing a natural shampoo by adding a caustic potash, including: washing herbs including 6 to 9 weight % of lotus roots, 4 to 6 weight % of lotus leaves, 4 to 6 weight % of red ginseng, 4 to 6 weight % of Hippophae rhamnoides leaves and trunk, 4 to 6 weight % of persimmon leaves, 4 to 6 weight % of thuja orientalis, 4 to 6 weight % of ginger, 3 to 5 weight % of rosemary, 6 to 9 weight % of black beans, 3 to 5 weight % of black sesame, 5 to 8 weight % of aloe, 5 to 8 weight % of mulberry leaves, 1 to 3 weight % of Orostachys japonicus, 4 to 6 weight % of Angelica gigas, 3 to 5 weight % of green tea, 5 to 8 weight % of pine needles, 4 to 6 weight % of mint, 4 to 6 weight % of pleuropterus multflorus, 3 to 5 weight % of blueberry, 3 to 5 weight % of Acorus gramineus and 3 to 5 weight % of garlic, putting the washed herbs in a maturing device, and maturing the herbs at temperatures of 65 to 95° C. for 3 to 21 days depending on the herb (S100); putting the matured herbs along with 15 to 25 weight % of distilled water and 75 to 85 weight % of soft water in a decoction maker, boiling the herbs mixed with the water by strong heat, and decocting the herbs mixed with the water by weak heat for 48 to 72 hours so as to make a first decoction (S200); pouring the first decoction in a vessel, putting 37 to 43 weight % of distilled water or soft water in the decoction maker, boiling the herbs mixed with the water by strong heat and decocting the herbs mixed with the water by weak heat for 3 to 4 hours so as to make a second decoction (S300); removing the herbs from the decoction maker, mixing the first decoction with the second diction, and decocting the mixed decoction for 24 to 36 hours by weak heat so as to obtain a herbal medicine mature decoction of 7.5 to 8.5 weight % (S400); slowing mixing 45 to 55 weight % of caustic potash (KOH) in 45 to 55 weight % of distilled water, and cooling the mixture to 75 to 85° C. (S500); heating 63 to 73 weight % of carrier oil, which is a mixture of at least two of coconut oil, olive oil, camellia oil, and castor oil to 75 to 85° C., slowly pouring the heated carrier oil in 27 to 37 weight % of the mixture of cooled caustic potash and distilled water, and stirring the mixture so as to be saponificated (S600); mixing 10 to 20 weight % of sugar with 80 to 90 weight % of distilled water, and stirring the mixture and heating the mixture up to 80 to 90° C. (S700); mixing 20 to 30 weight % of the sugared water with 70 to 80 weight % of the saponificated mixture, and stirring the mixture (S800); pouring the saponificated mixture and the mixture of sugared water in a sealed container, and maturing the mixture in a shady and well-ventilated place in room temperatures of 22 to 28° C. for 1 to 3 weeks so as to obtain a natural shampoo paste (S900); mixing 43 to 53 weight % of the herbal medicine mature decoction prepared in the above step 4 (S400) with 47 to 57 weight % of the natural shampoo paste matured in room temperatures, and heating the mixture to 85 to 95° C. and stirring the mixture for 12 to 36 hours (S1000); obtaining 12.5 to 15 weight % of Effective Micro-organisms (EM) fermented liquid by mixing 90.5 to 93.5 weight % of water from washing rice, 1 to 1.5 weight % of sugar, 1 to 1.5 weight % of salt, 1 to 1.5 weight % of molasses, 1 to 1.5 weight % of original EM liquid, 1 to 1.5 weight % of EM activation liquid, and 1.5 to 2 weight % of rosemary and then sealing the mixture and fermenting the mixture in temperatures of 30 to 35° C. for 5 to 10 days, then adding the EM fermented liquid to 85 to 87.5 weight % of the mixture of the herbal medicine mature decoction and the natural shampoo paste and stirring the mixture (S1100); adding 0.1 to 5 weight % of salt to 95 to 99.9 weight % of mixture of the herbal medicine mature decoction, to which EM fermented liquid has been added, and then stirring the mixture in temperatures of 80 to 95° C. (S1200); adding 1 to 2 weight % of vitamin E and silk amino acid to 98 to 99 weight % of viscosity-increased mixture, and stirring the mixture (S1300); adding 1 to 6 weight % of essential oil, which is the mixture of at least two of lavender, lemon, clary sage, ylang ylang, rosemary, tea tree, and cedarwood, to the 94 to 99 weight % of the saponificated mixture, and then stirring the mixture (S1400); and pouring the mixture of the herbal medicine mature decoction, to which essential oil has been added, and the natural shampoo paste in a sealed container, and maturing the mixture in room temperatures of 22 to 28° C. in a shady and well-ventilated place for 5 to 10 days so as to obtain natural shampoo (S1500).

### BRIEF DESCRIPTION OF THE DRAWINGS

The above and other features and advantages of the present invention will become more apparent by describing in detail exemplary embodiments thereof with reference to the attached drawings in which:

FIG. 1 is a process diagram illustrating a method of preparing a natural shampoo according to the present invention; and

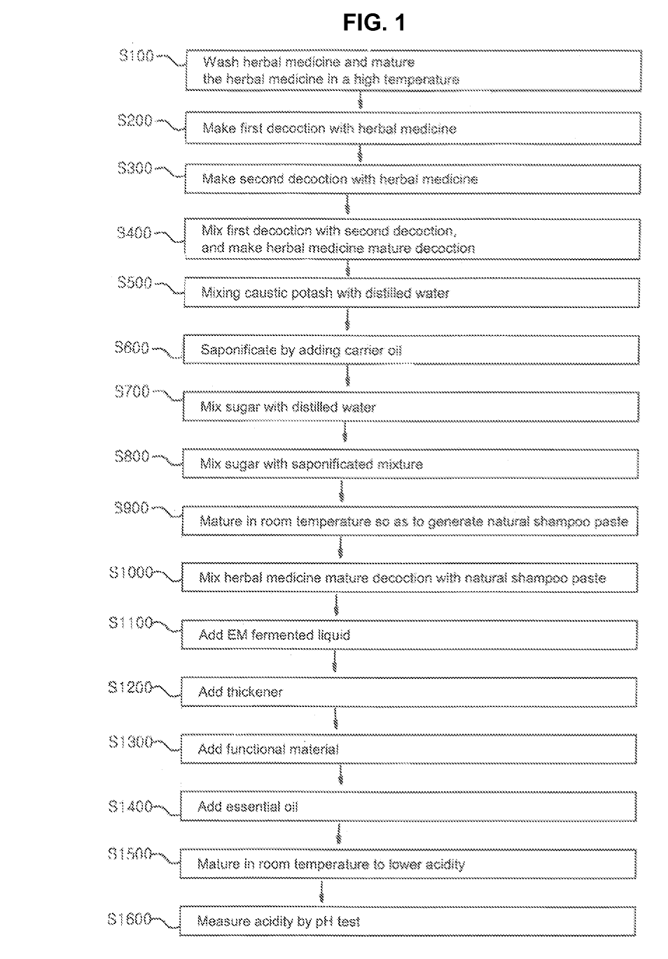

FIG. 2 is a questionnaire for checking the effects of a natural shampoo according to the present invention.

### DETAILED DESCRIPTION OF THE INVENTION

Exemplary embodiments of the present invention are described with reference to the accompanying drawings in detail. The same reference numbers are used throughout the drawings to refer to the same or like parts. Detailed descriptions of well-known functions and structures incorporated herein may be omitted to avoid obscuring the subject matter of the present invention.

### Exemplary embodiment 1

First, step 1 (S100) to step 4 (S400) are the process of making a herbal medicine mature decoction by high temperature maturing, and step 5 (S500) to step 16 (S1600) are the process of making a natural shampoo using the prepared herbal medicine mature decoction.

The features of the present invention to solve the problem of the conventional art are found in the process of making a herbal medicine mature decoction by high temperature maturing from step 1 (S100) to step 4 (S400), and the process from step 5 shows a process of preparing a natural shampoo using the herbal medicine mature decoction.

Step 1 (S100): Washing Herb Medicines and Maturing them in a High Temperature

Herbs having excellent effects on prevention of hair loss or stimulation of hair growth are chosen, washed in water, and matured in a high temperature so that a natural shampoo has excellent effects on prevention of hair loss or stimulation of hair growth.

Herbs selected in the present invention are 6 to 9 weight % of lotus roots, 4 to 6 weight % of lotus leaves, 4 to 6 weight % of red ginseng, 4 to 6 weight % of Hippophae rhamnoides leaves and trunk, 4 to 6 weight % of persimmon leaves, 4 to 6 weight % of thuja orientalis, 4 to 6 weight % of ginger, 3 to 5 weight % of rosemary, 6 to 9 weight % of black beans, 3 to 5 weight % of black sesame, 5 to 8 weight % of aloe, 5 to 8 weight % of mulberry leaves, 1 to 3 weight % of Orostachys japonicus, 4 to 6 weight % of Angelica gigas, 3 to 5 weight % of green tea, 5 to 8 weight % of pine needles, 4 to 6 weight % of mint, 4 to 6 weight % of pleuropterus multflorus, 3 to 5 weight % of blueberry, 3 to 5 weight % of Acorus gramineus and 3 to 5 weight % of garlic. The selected herbs are washed in water, and are placed in a maturing device and are matured for 3 to 21 days at 65 to 95° C. depending on the material.

The maturing device is a device developed by the present applicant to mature herbs as mentioned in Korean Patent No. 1128299, and is a device which is shut tightly not to allow a leak of water of the herbs, and maintains the constant temperature.

The setting temperature of the maturing device is preferably between 80 and 90° C., and the maturing period is differentially about 3 to 21 days.

The present applicant has performed an experiment of maturing herbs in a high temperature based on the fact that the remedial effects significantly increase when ginseng is steamed at a high temperature without boiling it. Further, through the first step (S100) of maturing herbs at a high temperature, unpleasant smell of herbs is reduced, the remedial effects significantly increase, and the matured herbs may be stored for a long time and may be mixed well with each other and with carrier oil when making a decoction, thereby allowing production of shampoo. Hence, in the present invention, step 1(S100) has most important technical features.

Among the herbal medicine, the lotus root has a lot of vitamin C protected by starch, and thus the vitamin C is not easily destroyed. As such, the lotus root has excellent effects of preventing pimples, freckles, and hair loss and promoting hair growth. Further, tannin helps blood circulation, and linolenic acid is a natural antioxidant and has effects of preventing aging. Further, mineral supplies nutrition to the scalp.

The remedial effects of the lotus leaves are similar to those of the lotus roots, but the lotus leaves especially have effects of quenching the thirst, soothing skin, reinforcing vigor, and detoxifying nicotine.

Red ginseng is a food for nutrition and robustness, and has a lot of effects such as fatigue recovery, immunity enhancement, platelet aggregation inhibition, hangover mitigation, aging prevention, high blood pressure and diabetes prevention, menopausal symptom improvement, and anemia prevention.

Hippophae rhamnoides leaves and trunk contain 15 kinds of vitamins, natural mineral, amino acids, saponin (4 times of ginseng), and have effects of treating high blood pressure, nervous prostration, heart disease, gestroenteric disorder, and eczema, and have anticancer effects, and effects of aging prevention and promotion of metabolism.

Persimmon leaves contains vitamin C (20 times of lemon), and has excellent effects on high blood pressure, scurvy, anemia, fatigue, and skin care.

Thuja orientalis remove unpleasant smell, make hair black, strengthen teeth and bones, have effects on treating or preventing hepatocirrhosis and liver cancer, and have effects on preventing high blood pressure and paralysis.

Ginger detoxifies the toxic elements of herbs and warms the body, and has effects of preventing hair loss and promoting hair growth.

Rosemary has excellent sterilizing power, has effects of aging prevention, calming effects, anti-oxidation effects, is used in a tea or a drink, and has effects of preventing hair loss and promoting hair growth.

Black beans normalize blood pressure, treats inflammation, and vitamin E, unsaturated fatty acids, anthocyanin, and beta carotene of the beans prevent aging. Further, estrogens, saponin, glycinin, etc. of the beans enlarge blood vessels, thereby relieving white hair and hair loss.

Black sesames contain unsaturated fatty acids which prevent hardening of the arteries, vitamin E and phospholipids which recover skin, and lecithin which is good to the brain, supplies nutrition to the scalp, thereby preventing hair loss and being helpful in anti-constipation.

Aloe improves fine wrinkles and scars, has antibiotic and anti-virus effects, cools the skin heat, relieves sensitive skin, has good moisturizing effects, heals and regenerates cells, washes, regenerates and moisturizes the scalp, and prevents hair loss.

Gava of mulberry leaves lowers blood pressure, strengthens the capillary vessel, thereby promoting hair growth, and has effects of preventing or healing apoplexy, high blood pressure, hardening of the arteries, and the heart disease.

Orostachys japonicus grows in an old roofing tile, and has excellent effects on cell activation, DNA immunity enhancement, detoxication, liver function recovery, and regeneration and recovery of damaged cells.

Angelica gigas improve the circulation of blood, strengthen the heart function, mitigates the cold symbols of hands and foot, prevents aging, improves the scalp condition and promotes hair growth.

Green teas contain a lot of catechin having strong anti-cancer effects, inhibits inflammation and germ infection, inhibits the rise of the blood sugar level, prevents aging, detoxifies nicotine and heavy metal, recovers hangover and fatigue, changes acidic constitution, removes carious teeth and bad breath, and improves the skin state.

Pine needles contain chlorophyll, vitamin A, and vitamin C, has curative effects on apoplexy, high blood pressure, hardening of the arteries, haemorrhoids, dermatitis, kidney disease, anemia, and neuralgia, and regenerates cells, prevents aging, prevents hair loss and promotes hair growth.

Mints contain a lot of manganese, vitamin A and vitamin C, and thus has antibiotic effects, cools the heat, removes the skin damage and inflammation, invigorates the person, relieves the itching, promotes the blood circulation, reduces pores, and has curative effects on pimples and dermatitis.

Pleuropterus multflorus enlarges blood vessels, regenerates skin cells, and has effects on hair gloss, white hair prevention, hair loss prevention and hair growth promotion.

Blueberries are one of the world's top 10 health foods, and strengthens eyesight, ha anticancer effects, prevents aging, and has curative effects on constipation, osteoporosis, etc.

Acorus gramineus clears the head, improves memory, prevents aging, and makes hair glossy and maintains the health of hair.

Garlic contains allicin that has sterilizing and anti-bacterial effects and effects of activating cells and promoting blood circulation which are better than the effects of penicillin and terramycin, and contains germanium that is helpful in fatigue recovery and physical strength enhancement when combined with vitamin B1, thereby being helpful in hair growth.

Step 2 (S200): Making a First Decoction Using Herbal Medicines

Herbs, which have been matured in a high temperature, have significantly reduced unpleasant smell, and may now be mixed with each other, are put in a decoction maker along with 15 to 25 weight % of distilled water and 75 to 85 weight % of soft water, then they are boiled with strong heat, then they are boiled with weak heat for 48 to 72 hours so as to make the first decoction.

Water put in the decoction maker along with herbs should be distilled water and soft water, not tap water. The reason why distilled water and soft water should be used is because tap water contains materials which have been used at the time of chemical treatment and the materials lower the curative effects of the herbal medicines, and when the materials are reacted with caustic potash, unusual reaction occurs.

Step 3 (S300): Making a Second Decoction with Herbal Medicines

The first decoction made in step 2 (S200) is poured in another vessel, and 37 to 43 weight % of distilled water or soft ware is poured in the original vessel, then the water in the original vessel is boiled by strong heat, and is then boiled by weak heat for 3 to 4 hours so as to make the second decoction.

Step 4 (S400): Mixing the First Decoction with the Second Decoction and Boiling the Mixed Decoction to Make a Herbal Medicine Mature Decoction

After removing the herbal medicines from the second decoction vessel, the first decoction is mixed with the second decoction, and then the mixed decoction is boiled with weak heat for 24 to 36 hours so as to obtain a herbal medicine mature decoction of 7.5 to 8.5 weight %.

Through the process of making the first decoction and the second decoction using the herbal medicines, and making the herbal medicine mature decoction by mixing the first decoction and the second decoction and boiling the mixed decoction, the herbal medicines, which have been matured in a high temperature, now have significantly less unpleasant smell and may be mixed with each other.

Step 5 (S500): Mixing the Herbal Medicine Mature Decoction with Caustic Potash

45 to 55 weight % of caustic potash (KOH) is slowly mixed with 45 to 55 weight % of distilled water, and the mixture is cooled to 75 to 85° C.

The caustic potash reacts with the distilled water, and thereby emits gas and generates intense heat, and this is cooled down to 75 to 85° C. in the air.

Step 6 (S600): Adding Carrier Oil so as to be Saponificated

63 to 73 weight % of carrier oil, which is a mixture of at least two of coconut oil, olive oil, camellia oil, and castor oil, is heated to 75 to 85° C., and the heated carrier oil is slowly poured in 27 to 37 weight % of the mixture of cooled caustic potash and distilled water, and the mixture is stirred so as to be saponificated.

When the carrier oil is added to the mixture of cooled caustic potash and distilled water, the person in charge stirs them while slowly pouring the carrier oil in the mixture, and viscosity increases to the point when, if the final mixture is dropped, a mark remains. The point is called the trace state, and implies that saponification has been done well.

When all of the carrier oils are added, 39 to 49 weight % of coconut oil, 21 to 31 weight % of olive oil, 5 to 15% of camellia oil, and 15 to 25 weight % of castor oil may be used for 63 to 73 weight % of the carrier oil.

Step 7 (S700): Mixing Sugar with Distilled Water

10 to 20 weight % of sugar is mixed with 80 to 90 weight % of distilled water, and then the mixture is stirred and heated up to 80 to 90° C. so as to complete melt sugar.

Sugar is added to enhance the moisturizing effect and increase transparency and adding more than 20 weight % of sugar did not show any significant difference, and thus only 10 to 20 weight % of sugar is added.

Step 8 (S800): Mixing Sugared Water with Saponificated Mixture

20 to 30 weight % of the sugared water is mixed with 70 to 80 weight % of the saponificated mixture, and the mixture is stirred.

Step 9 (S900): Maturation in the Room Temperature for Sufficient Generation of Natural Nutrients

The saponificated mixture and the mixture of sugared water are poured in a sealed container, and the mixture is then matured in room temperatures of 22 to 28° C. for 1 to 3 weeks so that natural nutrients are abundantly generated.

Through the maturation in room temperatures, the oil (carrier oil) and water (distilled water) is mixed well and natural nutrients are generated, and the mixture becomes transparent.

The step is progressed in a completely sealed state because, if the saponificated mixture continually contacts air, the mixture becomes hardened within a short period of time. The natural nutrients cannot be generated in the hardened state, and thus a long time maturation in room temperatures in the sealed state is progressed so that sufficient natural nutrients and glycerine may be generated before hardened.

Through the maturation in room temperatures, the ph (acidity) of the mixture is lowered and sufficient maturation is done. At this time, direct exposure to sunlight may destroy nutrients and change color, and thus the mixture is matured in room temperatures in a shady and well-ventilated place.

Step 10 (S1000): Mixing Herbal Medicine Mature Decoction with Natural Shampoo Paste

43 to 53 weight % of the herbal medicine mature decoction prepared in the above step 4 (S400) is mixed with 47 to 57 weight % of the natural shampoo paste matured in room temperatures, and the mixture is heated to 85 to 95° C. and is then stirred for 12 to 36 hours.

Step 11 (S1100): Adding EM Fermented Liquid to the Mixture of the Herbal Medicine Mature Decoction and the Natural Shampoo Paste

The prepared 12.5 to 15 weight % of Effective Micro-organisms (EM) fermented liquid is added to 85 to 87.5 weight % of the mixture of the herbal medicine mature decoction and the natural shampoo paste, and the mixture is stirred.

The EM fermented liquid has been generated by fermenting effective micro-organisms composed of 80 to 125 kinds of micro-organisms such as yeast, lactic acid bacteria, photosynthetic bacteria and aspergillus bacteria. Currently, EM fermented liquids manufactured in various methods have been developed and used.

The EM fermented liquid prepared by the present application has been generated by mixing 90.5 to 93.5 weight % of water from washing rice, 1 to 1.5 weight % of sugar, 1 to 1.5 weight % of salt, 1 to 1.5 weight % of molasses, 1 to 1.5 weight % of original EM liquid, 1 to 1.5 weight % of EM activation liquid, and 1.5 to 2 weight % of rosemary, then sealing the mixture and fermenting the mixture in temperatures of 30 to 35° C. for 5 to 10 days.

The original EM liquid is a liquid before effective micro-organisms are activated, and the EM activation liquid is a liquid acquired by activation of micro-organisms obtained by first fermentation of the original EM liquid. The present applicant tried to obtain a high-quality EM fermented liquid by promoting fermentation by adding already activated EM activation liquid as a result of a study of several months as well as the original EM liquid.

Furthermore, rosemary has excellent effects in preventing hair loss and promoting hair growth, and thus if rosemary is added when fermented, the fermented liquid may have the effects.

The EM fermented liquid prepared by the present applicant has effects in preventing hair loss and promoting hair growth because micro-organisms contained in the EM fermented liquid have excellent anti-oxidation, antibiotic, anti-allergic and anti-inflammatory effects and thus remove harmful bacteria that are parasitic on the scalp and stabilize the scalp.

Step 12 (S1200): Adding Salt to the Mixture of the Herbal Medicine Mature Decoction, to which EM Fermented Liquid has been Added, and the Natural Shampoo Paste

0.1 to 5 weight % of salt is added to 95 to 99.9 weight % of mixture of the herbal medicine mature decoction, to which EM fermented liquid has been added, and the natural shampoo paste, and the mixture is stirred in temperatures of 80 to 95° C.

If salt is added to the mixture, the salt is used as a natural preservative and thickener and thus increases viscosity of the mixture.

Step 13 (S1300): Adding Function Material to Mixture of Herbal Medicine Mature Decoction and Natural Shampoo Paste

1 to 2 weight % of vitamin E and silk amino acid are added to 98 to 99 weight % of viscosity-increased mixture, and the mixture is stirred.

Vitamin E may also be called tocopherol, and has effects in preventing dementia, arteriosclerosis, cancer and arthritis, preventing and treating cataract, protects cells, and maintains a normal structure and function of the body.

Furthermore, silk amino acid contains serine that is used to treat diabetes and diabetic complications, glycine that has good effects in treating low cholesterol in blood and diseases related with blood vessel, tyrosine that has effects in treating Alzheimer's disease and Parkinson's disease, alanine that has effects in fatigue recovery, and silk amino acid that has effects in treating atopic dermatitis, and silk has excellent effects in blocking ultraviolet rays, removing freckles, whitening and moisturizing skin.

Step 14 (S1400): Adding Essential Oil to Mixture of Herbal Medicine Natural Decoction and Natural Shampoo Paste

1 to 6 weight % of essential oil, which is the mixture of at least two of lavender, lemon, clary sage, ylang ylang, rosemary, tea tree, and cedarwood, is added to the 94 to 99 weight % of the saponificated mixture, and then stirred.

The essential oil is added because, even if most of unpleasant smell of natural herbs has been removed through the first decoction and the second decoction, but unique herbal medicine smell still remains. Hence, in order to remove the remaining smell and add functional features of each oil, the essential oil is added.

In the essential oil, lavender has strong fragrance and preservative effects, has curative effects on pimples, dermatitis, tumors, eczema, a bite by a bug, athlete's feet, ringworm, injury, and wrinkles, and has curative effects on stress, depression, insomnia, and headache.

Further, lemon helps fatigue recovery, blood circulation, metabolism activation, skin and mucous membrane reinforcement, and reinforcement of resistance to bacteria, and has effects on skin peeling of oily skin, injury recovery, and hair cleansing.

Further, clary sage has effects of preservation from decay, sterilization, tranquilization, prevention of unpleasant smell, and removal of fever, has effects on sensitive skin, stress, external wound, seborrheic dandruff, and promotes hair growth.

Further, ylang ylang has tranquilization effects, preservative effects, aphrodisiac effects, pimple treatment effects, normal blood pressure maintenance effects, pain killing effects, hair loss prevention effects, skin water balance regulation effects, and hair crack prevention effects.

Further, rosemary has cell restoration promotion and lymphatic drainage stimulation effects, has effects on skin aging by promoting generation of fibroblasts, has blood circulation improvement, antibiotic effects, pain killing effects, inflammation improvement, sexual desire reinforcement, and urination stimulation, gives vigor to brain cells by strong fragrance so as to have effects on memory and concentration improvement, dementia prevention and gloss and elastic skin maintenance, and has neuralgia and dandruff treatment effects, hair loss prevention and hair growth stimulation effects.

Further, the tea tree has effects on skin diseases by bacteria, has effects on pimples, eczema, oily skin, athlete's feet, wart, a bite by a bug, dandruff, and injury, and improves the immune system.

Further, cedarwood helps lymphatic drainage, decomposes cellulite, has effects on pimples, oily hair, dandruff and preservation from decay, has urination stimulation effects and sterilization effects so as to have the best effects on oily skin, thereby has pimple treatment effects, seborrhoeic dermatitis treatment effects and hair loss treatment effects, and is used in a hair tonic because of scalp cell detoxication effects.

Step 15 (S1500): Maturation of Mixture of Herbal Medicine Natural Decoction and Natural Shampoo Paste in a Room Temperature

The mixture of the herbal medicine mature decoction, to which essential oil has been added, and the natural shampoo paste are poured in a sealed container, and the mixture is matured in room temperatures of 22 to 28° C. in a shady and well-ventilated place for 5 to 10 days so as to obtain natural shampoo.

Step 16 (S1600): Measuring Acidity by pH Test

Natural shampoo, of which the maturing in a room temperature has been completed, is separated, and a pH test is performed to measure the acidity, and the acidity is checked to confirm whether the acidity is between neutrality and 9 (weak alkali) so as to complete the product.

If the acid shampoo is used because of insufficient maturation, the scalp turns red and prickles and the skin is injured, and thus the shampoo is matured in a room and warm temperature for sufficient maturation and improvement of the curative effects of the herbal medicine mature decoction, thereby producing high-quality shampoo that has excellent effects in preventing hair loss and promoting hair growth.

### Experimental Example 1

### Experiment of Efficacy of Natural Shampoo of the Embodiment of the Present Invention

Through the embodiment of the present invention, natural shampoo using the herbal medicine mature decoction is produced, and the efficacy of the shampoo is experimented by making men and women in their 30s to 60s use the shampoo for more than 1 month (30 days).

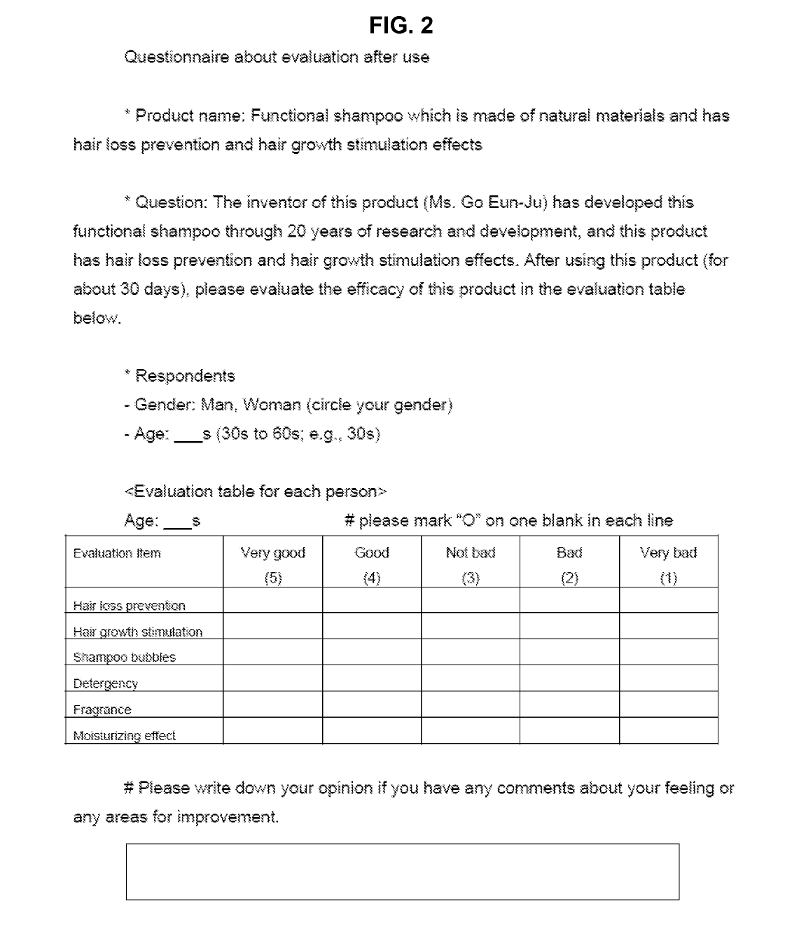

As shown in FIG. 2, “questionnaire after use of the product” is distributed, and the subjects evaluate the shampoo and record their evaluation according to the criteria disclosed in the questionnaire (Very good (5), Good (4), Not bad (3), Bad (2), Very bad (1)), and the result is shown in Table 1 below.

TABLE 1

| | | | | | |
|---|---|---|---|---|---|
| Evaluation Item | 30s | 40s | 50s | 60s | Average |

(Men)

| | | | | | |
|---|---|---|---|---|---|
| Hair loss prevention | 5 | 5 | 5 | 5 | 5 |
| Hair growth stimulation | 5 | 5 | 5 | 5 | 5 |
| Shampoo bubbles | 5 | 5 | 5 | 4.3 | 4.8 |
| Detergency | 5 | 5 | 5 | 5 | 5 |
| Fragrance | 3.9 | 4.4 | 4.3 | 3.5 | 4 |
| Moisturizing effect | 5 | 5 | 4.9 | 4.5 | 4.9 |

(Women)

| | | | | | |
|---|---|---|---|---|---|
| Hair loss prevention | 5 | 5 | 5 | 4.7 | 4.9 |
| Hair growth stimulation | 5 | 4.5 | 4.5 | 4.8 | 4.7 |
| Shampoo bubbles | 5 | 5 | 5 | 4.7 | 4.9 |
| Detergency | 5 | 5 | 5 | 5 | 5 |
| Fragrance | 3.8 | 4.5 | 4 | 4.3 | 4.1 |
| Moisturizing effect | 5 | 5 | 5 | 5 | 5 |

Referring to Table 1 above, most of men and women in 305 to 605 have marked “very good” for hair loss prevention, hair growth stimulation, shampoo bubbles, detergency and moisturizing effect, and marked “good” only for fragrance. This is because most of people including the subjects of this experiment have used products containing artificial fragrance. Because natural fragrance in conventional natural shampoo is weak, artificial fragrance has been added, but it may be harmful to the human body and may give detrimental effects on the natural elements, and thus this problem has been resolved by adjusting the amount of essential oil.

Referring to the comments of some of the subjects of this experiment, Ms. Kim, one woman in her 40s, said “My hair was full of dandruff, hair loss and itch was serious, but after using this shampoo, all of these problems have been improved . . . ” Ms. Choi, one woman in her 60s, said “Hair loss was so serious due to frequent dyeing and perm, but after using this shampoo, hair loss problem has been improved and my hair has become glossy.” Mr. Gil, one man in his 60s, said “whenever I wash my hair, I lost a lot of hairs, but 23 days after using the natural shampoo, the hair loss stopped and new hairs are started to be generated. Thank you.” Mr. Park, one woman in her 40s, said “I used this shampoo after I heard about the effects of hair loss prevention, and after I used it, hair loss was reduced and new hairs are started to be generated. Likewise, many people recognized the effects of the natural shampoo of the present invention.

In this way, a natural shampoo made of herbal medicines having excellent effects of hair loss prevention and hair growth stimulation has been developed, and according to the present invention, it has become possible to provide a natural shampoo using a herbal medicine mature decoction which may be easy to be used by significantly reducing unpleasant smell of natural herbs, may be mixed with various natural herbs by reducing the attribute of the natural herbs of being separated from an carrier oil, may be stored for a long time by doubling elements having medical effects of natural herbs, may prevent loss of hair and stimulate hair growth, and a method of preparing the same.

According to the present invention, there are effects of providing a natural shampoo prepared by using a herbal medicine mature decoction which may be easy to be used by significantly reducing unpleasant smell of natural herbs, may be mixed with various natural herbs by reducing the attribute of the natural herbs of being separated from an carrier oil, may be stored for a long time by doubling elements having medical effects of natural herbs, may prevent loss of hair and stimulate hair growth by using a herbal medicine mature decoction of Korean Patent No. 1128299 owned by the applicant of the present application, and a method of preparing the same

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A natural shampoo having functions of preventing hair loss and promoting hair growth, which is made using a herbal medicine mature decoction which is obtained by putting herbs including lotus roots, lotus leaves, red ginseng, Hippophae rhamnoides leaves and trunk, persimmon leaves, thuja orientalis, ginger, rosemary, black beans, black sesame, aloe, mulberry leaves, Orostachys japonicus, Angelica gigas, green tea, pine needles, mint, pleuropterus multflorus, blueberry, Acorus gramineus, and garlic in a maturing device, maturing the herbs in temperatures of 65 to 95° C. depending on the herb for 3 to 21 days, then pouring distilled water or soft water, and decocting the matured herbs mixed with the water.

2. The natural shampoo of claim 1, wherein the herbal medicine mature decoction is obtained by putting the herbs matured in the temperatures along with the distilled water or soft water in a decoction maker, then boiling the herbs mixed with the water by strong heat, then decocting the herbs mixed with the water by weak heat so as to make a first decoction, then pouring the first decoction in another vessel, then pouring distilled water or soft water in the decoction maker, then boiling the herbs mixed with the water by strong heat, then decoct the herbs mixed with the water by weak heat so as to make a second decoction, then removing the herbs from the second decoction, then mixing the first decoction with the second decoction, and then decocting the mixed decoction by weak heat.

3. A method of preparing a natural shampoo by adding a caustic potash, the method comprising:
washing herbs including 6 to 9 weight % of lotus roots, 4 to 6 weight % of lotus leaves, 4 to 6 weight % of red ginseng, 4 to 6 weight % of Hippophae rhamnoides leaves and trunk, 4 to 6 weight % of persimmon leaves, 4 to 6 weight % of thuja orientalis, 4 to 6 weight % of ginger, 3 to 5 weight % of rosemary, 6 to 9 weight % of black beans, 3 to 5 weight % of black sesame, 5 to 8 weight % of aloe, 5 to 8 weight % of mulberry leaves, 1 to 3 weight % of Orostachys japonicus, 4 to 6 weight % of Angelica gigas, 3 to 5 weight % of green tea, 5 to 8 weight % of pine needles, 4 to 6 weight % of mint, 4 to 6 weight % of pleuropterus multflorus, 3 to 5 weight % of blueberry, 3 to 5 weight % of Acorus gramineus and 3 to 5 weight % of garlic, putting the washed herbs in a maturing device, and maturing the herbs at temperatures of 65 to 95° C. for 3 to 21 days depending on the herb (S100);
putting the matured herbs along with 15 to 25 weight % of distilled water and 75 to 85 weight % of soft water in a decoction maker, boiling the herbs mixed with the water by strong heat, and decocting the herbs mixed with the water by weak heat for 48 to 72 hours so as to make a first decoction (S200);
pouring the first decoction in a vessel, putting 37 to 43 weight % of distilled water or soft water in the decoction maker, boiling the herbs mixed with the water by strong heat and decocting the herbs mixed with the water by weak heat for 3 to 4 hours so as to make a second decoction (S300);
removing the herbs from the decoction maker, mixing the first decoction with the second diction, and decocting the mixed decoction for 24 to 36 hours by weak heat so as to obtain a herbal medicine mature decoction of 7.5 to 8.5 weight % (S400);
slowing mixing 45 to 55 weight % of caustic potash (KOH) in 45 to 55 weight % of distilled water, and cooling the mixture to 75 to 85° C. (S500);
heating 63 to 73 weight % of carrier oil, which is a mixture of at least two of coconut oil, olive oil, camellia oil, and castor oil to 75 to 85° C., slowly pouring the heated carrier oil in 27 to 37 weight % of the mixture of cooled caustic potash and distilled water, and stirring the mixture so as to be saponificated (S600);
mixing 10 to 20 weight % of sugar with 80 to 90 weight % of distilled water, and stirring the mixture and heating the mixture up to 80 to 90° C. (S700);
mixing 20 to 30 weight % of the sugared water with 70 to 80 weight % of the saponificated mixture, and stirring the mixture (S800);
pouring the saponificated mixture and the mixture of sugared water in a sealed container, and maturing the mixture in a shady and well-ventilated place in room temperatures of 22 to 28° C. for 1 to 3 weeks so as to obtain a natural shampoo paste (S900);
mixing 43 to 53 weight % of the herbal medicine mature decoction prepared in the above step 4 (S400) with 47 to 57 weight % of the natural shampoo paste matured in room temperatures, and heating the mixture to 85 to 95° C. and stirring the mixture for 12 to 36 hours (S1000);
obtaining 12.5 to 15 weight % of Effective Micro-organisms (EM) fermented liquid by mixing 90.5 to 93.5 weight % of water from washing rice, 1 to 1.5 weight % of sugar, 1 to 1.5 weight % of salt, 1 to 1.5 weight % of molasses, 1 to 1.5 weight % of original EM liquid, 1 to 1.5 weight % of EM activation liquid, and 1.5 to 2 weight % of rosemary and then sealing the mixture and fermenting the mixture in temperatures of 30 to 35° C. for 5 to 10 days, then adding the EM fermented liquid to 85 to 87.5 weight % of the mixture of the herbal medicine mature decoction and the natural shampoo paste and stirring the mixture (S1100);
adding 0.1 to 5 weight % of salt to 95 to 99.9 weight % of mixture of the herbal medicine mature decoction, to which EM fermented liquid has been added, and then stirring the mixture in temperatures of 80 to 95° C. (S1200);
adding 1 to 2 weight % of vitamin E and silk amino acid to 98 to 99 weight % of viscosity-increased mixture, and stirring the mixture (S1300);
adding 1 to 6 weight % of essential oil, which is the mixture of at least two of lavender, lemon, clary sage, ylang ylang, rosemary, tea tree, and cedarwood, to the 94 to 99 weight % of the saponificated mixture, and then stirring the mixture (S1400); and
pouring the mixture of the herbal medicine mature decoction, to which essential oil has been added, and the natural shampoo paste in a sealed container, and maturing the mixture in room temperatures of 22 to 28° C. in a shady and well-ventilated place for 5 to 10 days so as to obtain natural shampoo (S1500).
